(19) United States
(12) Patent Application Publication
(10) Pub. No.: US 2025/0312257 A1
(43) Pub. Date: Oct. 9, 2025

(54) Surfactant Concentrates

(71) Applicant: Vantage Specialty Ingredients, Inc., Warren, NJ (US)

(72) Inventors: Xiang Chen, Somerset, NJ (US); Michael J. Fevola, Belle Mead, NJ (US); Phatcharada Phondee, Morristown, NJ (US); Daniel A. Tempesta, Milford, CT (US)

(73) Assignee: Vantage Specialty Ingredients, Inc., Warren, NJ (US)

(21) Appl. No.: 19/170,181

(22) Filed: Apr. 4, 2025

Related U.S. Application Data
(60) Provisional application No. 63/574,489, filed on Apr. 4, 2024.

(51) Int. Cl. A61K 8/46; A61Q 5/02; A61Q 5/12

(52) U.S. Cl. CPC A61K 8/466; A61Q 5/02; A61Q 5/12

(57) ABSTRACT

A sulfate-free surfactant concentrate consisting essentially of (I) at least one, preferably two, acyl N-alkyl taurate(s), or salt(s) thereof; (ii) an acyl N-alkyl aminoalkanoate, or salt thereof; and (iii) water. The molar ratio of the acyl N-alkyl aminoalkanoate to the acyl N-alkyl taurate(s) is from about 1:1 to about 3:1. The concentrate is substantially free of any surfactant that is comprised of a sulfate moiety and/or a hydrogen bond donating amide moiety.

### BACKGROUND OF THE INVENTION

Consumer perception of cleansing products for hair, scalp, body, and facial skin is based on a multiplicity of sensory cues: overall foam; quantity and quality of flash foam; creaminess of lather; time required to generate a “good” lather; wetting and spreading of lather; rinseability and afterfeel (including residue left after rinsing). In the case of shampoos and conditioners, shine, and manageability (including wet combing and dry combing) are important considerations.

Surfactants bearing anionic sulfate ester head groups, herein referred to as “sulfates”, have long been used in personal care formulations to create stable, long-lasting foam, and provide desirable viscosity. However, if not carefully formulated for mildness, sulfates can exhibit greater levels of skin and eye irritation, as well as skin drying tendencies. In response to irritancy, drying, and other concerns (e.g. fading in color-treated hair), an increasing number of sulfate-free cleansing products have become available.

Formulating sulfate-free products presents a multitude of challenges. Generating lather profiles similar to sulfate systems can require higher overall loadings of “milder” non-sulfate surfactants, increasing costs and potential for irritancy. Moreover, certain surfactants can strip hair of its natural oils, not only exacerbating dryness, but leading to reduced shine, increased breakage, split ends, and frizz. When hair fibers are not in parallel alignment—as is often the case with the damaged and/or dry hair—light reflectance decreases, light scattering increases resulting in reduced luster (dullness). Additionally, achieving higher viscosities in sulfate-free systems is known to be difficult. These challenges are compounded when seeking to formulate clear (substantially transparent or translucent) products and striving to meet “clean” beauty certification standards that require formulations “free of” an ever-increasing list of ingredients. For example, Cocamide DEA, a thickening agent that also serves as an effective foam builder and foam stabilizer, is avoided due to concerns from listing on California Proposition 65.

Alkyl taurate amides and taurate salts are anionic surfactants used in formulating mild sulfate-free cleansing formulations. They can not only create rich dense foam but can also leave a soft afterfeel after rinsing. However, taurates are typically available in the form of pastes or solids, which require heating during manufacturing and can be difficult to handle in large scale manufacturing operations without specialized equipment.

Expired Canadian Patent 1,330,924 (“the '924 Patent”) teaches a liquid detergent composition comprising at least one taurate surfactant (from 0.5% to 10%), at least one sarcosinate surfactant (from 0.5% to 10%), and at least one auxiliary surfactant defined to include salts of sulfate esters of polyethylene glycol ethers of fatty alcohols, alkyl sulfate surfactants, and mixtures thereof (from 1% to 30%). The '924 Patent further teaches the weight ratio of taurate surfactant to sarcosinate surfactant of from about 3:1 to about 1:3, and a total surfactant level of from about 2% to about 40%. A composition containing sodium methyl cocoyl taurate and sodium lauroyl sarcosinate, in ratios of from about 3:1 to 1:3, each at concentration of from 0.5% to 10% is disclosed. Paste compositions containing even higher loadings of surfactants are taught to contain at least one taurate surfactant (from 1% to 20%), at least one sarcosinate surfactant (from 1% to 20%), and at least one auxiliary surfactant defined to include salts of sulfate esters of polyethylene glycol ethers of fatty alcohols, alkyl sulfate surfactants, and mixtures thereof (from 3% to 50%).

US Pre-Grant Patent Application Publication 2024/0299272 discloses a detersive surfactant comprising (a) an acyl taurate surfactant alone or in combination with an N-alkyl acyl taurate surfactant, (b) an amphoteric co-surfactant, and (c) water. The ratio a:b of detersive surfactant to co-surfactant is from about 0.5:1 to about 2:1.

Acyl N-alkyl taurate surfactants (and their salts) are sometimes combined in a formulation-for example, sodium methyl cocoyl taurate in combination with sodium methyl oleoyl taurate. Acyl N-alkyl taurate surfactants (and their salts) are also sometimes formulated in combination with sarcosinates, including sodium lauroyl sarcosinate. Some commercially available products include all three of sodium methyl cocoyl taurate, sodium methyl oleoyl taurate, and sodium lauroyl sarcosinate:
ONLY SKIN™ foaming face and body cleanser (https://www.onlyskin.com/products/cleanser);
SOAP & GLORY® Get A Smooth On™ (marketed as sulphate free for dull/frizzy hair types); https://web.archive.org/web/20200926152303/https://www.soapandglory.com/get-a-smooth-ontm-873);
TRUE FROG™ Anti-Hairfall Shampoo (https://truefrog.in/products/anti-hairfall-shampoo-everyday-hair-conditioner-combo-250ml-250 ml);
BULL DOG® Peppermint & Eucalyptus Shower Gel (https://uk.bulldogskincare.com/products/peppermint-eucalyptus-shower-gel)

(All of the above-referenced websites were accessed Mar. 25, 2024.)

Importantly, the four commercial products described in the immediately preceding paragraphs, like the compositions taught in the '924 Patent, include one or more additional surfactants.

The ONLY SKIN™ face and body cleanser contains Disodium Cocoamphodiacetate, Cocamidopropyl Hydroxysultaine, Sodium Lauroyl Methyl Isethionate, Coco Glucoside and Lauryl Glucoside.

The SOAP & GLORY® shampoo contains Sodium Lauroyl Methyl Isethionate and Cocamidopropyl Betaine.

The TRUE FROG™ shampoo contains Cocamidopropyl Betaine and Sodium Cocoyl Isethionate.

The BULL DOG shampoo contains Cocamidopropyl Betaine and Lauryl Glucoside.

Each of the ONLY SKIN™, SOAP & GLORY®, TRUE FROG™ and BULL DOG® products contains a zwitterionic surfactant with a hydrogen bond donating amide moiety—Cocamidopropyl Betaine (CAPB) or Cocamidopropyl Hydroxysultaine (CAPHS). CAPB, CAPHS and other surfactants with a hydrogen bond donating amide moiety are not part of the inventive surfactant concentrates of the present invention.

By increasing the number and/or loading of surfactants in a formulation, irritation potential increases. Thus, there has been and remains a long-felt but unmet need for surfactant systems with less, not more, surfactants.

There is also a need for sulfate-free, cold-process surfactant systems that are free-flowing (having a viscosity of less than about 1,000 cps) and clear (substantially transparent and free of haze or cloudiness), which can be mixed with other ingredients to make finished formulations without heating. Such cold-process surfactant systems provide economic and environmental benefits from reduced energy consumption.

Surprisingly and unexpectedly, cleansing compositions, in particular shampoos, made with concentrates of the present invention are able to provide similar foaming to sulfate systems as well as other desirable sensory benefits (including softness and combability), frizz control, and shine, whereas skin cleansers prepared from the concentrates provide improved skin hydration.

### SUMMARY OF THE INVENTION

A surfactant concentrate (SC) comprising or consisting essentially of (i) at least one, preferably two, acyl N-alkyl taurate(s) (or salts thereof), preferably acyl N-methyl taurates; (ii) an acyl N-alkyl aminoalkanoate (or salt thereof), preferably a sarcosinate; and (iii) water, wherein the molar ratio of the acyl N-alkyl aminoalkanoate to the acyl N-alkyl taurate(s) is from about 1:1 to about 3:1. The SC preferably contains a first acyl N-alkyl taurate that is a C₈-C₁₈ saturated acyl N-alkyl taurate, preferably sodium methyl cocoyl taurate. The SC may, and preferably does, contain a second acyl N-alkyl taurate that is a C₁₆-C₂₂ unsaturated or branched acyl N-alkyl taurate, preferably sodium methyl oleoyl taurate. The SC is substantially free not only of surfactants comprising a sulfate moiety but also surfactants comprising a hydrogen bond donating amide moiety.

Sulfate-free cleansing and cleansing-conditioning compositions (i.e., finished consumer products) for application to hair, skin or scalp containing the SC of the present invention provide one or more of the following benefits: easier combability (dry and wet); reduced frizz/volume control; reduced hair breakage; reduced split ends; increased hair fiber strength; increased luster/shine; reduced hair cuticle damage; improved hair surface quality (shine and smoothness); improved skin hydration; improved skin barrier function/reduced trans-epidermal water loss.

### BRIEF DESCRIPTION OF DRAWINGS

FIGS. 1 and 2 present the results of Example 3 and compare foamability-foam height and bubble count-generated by a common sulfate surfactant (sodium laureth sulfate and cocamidopropyl betaine) and the surfactant concentrate of the present invention.

FIG. 3 presents the results of Applications Example 4 and compare the combability (wet and dry) of the surfactant concentrate of the present invention versus other surfactants.

FIG. 4 presents the results of Applications Example 5 and shows reduction in frizz after application of a 9% solution of a surfactant concentrate according to the present invention (referenced as METAUPON® AmiBio™).

FIG. 5 presents the results of Applications Example 6 and compares the hydration of the surfactant concentrate of the present invention versus sodium lauryl ether sulfate.

FIG. 6 presents the results of Applications Example 7 and compares sensory attributes of combability (wet and dry), softness (wet and dry), frizz control, and shine of the surfactant concentrate of the present invention versus a sulfate system.

FIGS. 7A and 7B are, respectively, photographs of sulfate-free surfactant concentrates in accordance with the invention (SC-1 to SC-4) and comparative examples (Comp-1 to Comp-5).

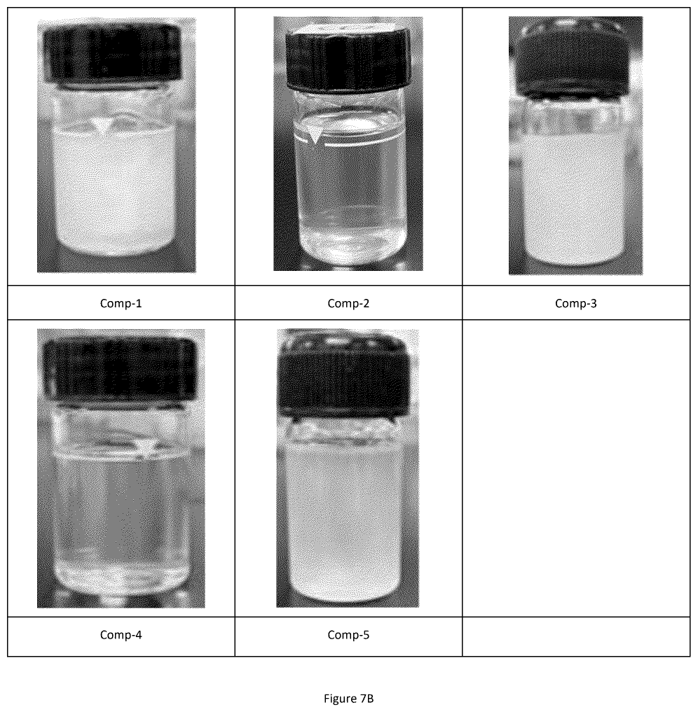

### DETAILED DESCRIPTION OF THE INVENTION

A first aspect of the present invention is directed to a SC comprising (i) at least one, preferably two, acyl N-alkyl taurates, preferably acyl N-methyl taurates; (ii) a C₈-C₁₈ saturated acyl N-alkyl aminoalkanoate or salt thereof, preferably a sarcosinate; and (iii) water, wherein the molar ratio of the acyl acyl N-alkyl aminoalkanoates to the acyl N-alkyl taurate(s) is from about 1:1 to about 3:1.

A second aspect of the present invention is directed to a SC consisting essentially of (i) at least one, preferably two, acyl N-alkyl taurates, preferably acyl N-methyl taurates; (ii) a C₈-C₁₈ saturated acyl N-alkyl aminoalkanoate or salt thereof, preferably a sarcosinate; and (iii) water, wherein the molar ratio of the acyl acyl N-alkyl aminoalkanoates to the acyl N-alkyl taurate(s) is from about 1:1 to about 3:1.

A third aspect of the present invention is directed to a SC consisting of (i) at least one, preferably two, acyl N-alkyl taurates, preferably acyl N-methyl taurates; (ii) a C₈-C₁₈ saturated acyl N-alkyl aminoalkanoate or salt thereof, preferably a sarcosinate; and (iii) water, wherein the molar ratio of the acyl acyl N-alkyl aminoalkanoates to the acyl N-alkyl taurate(s) is from about 1:1 to about 3:1

In the context of the present invention, taurate is to be understood as having a 2-aminoethane-1-sulfonate core.

By “substantially free of” is meant less than about 1%, preferably less than about 0.5%, more preferably less than about 0.1%, even more preferably less than about 0.05%, and most preferably less than about 0.01% of an intentionally added compound. In the context of the present invention the intentionally added compound is a surfactant comprising a sulfate moiety or a hydrogen bond donating amide moiety.

A first required component of a SC in accordance with the first, second or third aspect of the present invention is an acyl N-methyl taurate that is a C₈-C₁₈ saturated acyl N-alkyl taurate (or salt thereof) conforming to the structure

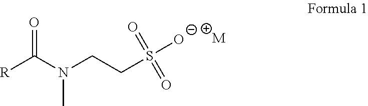

where RCO is a C₈-C₁₈ acyl moiety and M⁺ is a monovalaent cation. M⁺ may be ammonium (NH₄⁺, mono-, di-, or trisubstituted ammonium (i.e. RNH₃⁺, R₂NH₂⁺, R₃NH⁺), sodium, potassium, or a protonated amino acid salt such as sodium taurine or sodium methyl taurine.

The acyl moiety of C₈-C₁₈ saturated acyl N-alkyl taurates (and their salts) is preferably selected from the group consisting of caproyl, lauroyl, myristoyl, palmitoyl, stearoyl, and cocoyl.

In particularly preferred embodiments, the acyl group of the C₈-C₁₈ saturated acyl N-alkyl taurates is preferably cocoyl.

The International Cosmetic Ingredient Dictionary & Handbook published by the Personal Care Products Council (“PCPC”) defines coconut oil as the fixed oil obtained by expression from the kernel or seed of Cocos nucifera. Like other naturally-occuring triglyceride oils, coconut oil is a mixture of fatty acids of varying chain lengths.

In its “Safety Assessment of Alkyl Taurate Amides and Taurate Salts as Used in Cosmetics” issued on Feb. 8, 2016, the Cosmetic Ingredient Review (“CIR”) Expert Panel of the PCPC explains that “ingredients with a ‘cocoyl’ name are the result of reaction with coconut acid, which has a known composition of approximately: 0-1% caproic, 5%-9% caprylic, 6%-10% capric, 44%-52% lauric, 13%-19% myristic, 0-1% palmitoleic, 1%-3% stearic, 5%-8% oleic, and trace-2.5% linoleic acid.” http://www.cir-safety.org/sites/default/files/taurat122015FR.pdf at page 2 (accessed Mar. 26, 2025) (citing Elder, RL. Final report on the safety assesment of coconut oil, coconut acid, hydrogenated coconut acid, and hydrogenated coconut oil. Journal of the American College of Toxicology. 1986;5(3):103-121).

As used in the present application, the term “cocoyl” is to be understood to mean a mixture of fatty acids derived from coconut oil, or having at least 80% similarity to the following distribution of fatty acids, although not necessarily derived from coconut oil: caproic acid—up to about 1%; caprylic acid—from about 4% to about 9%; capric acid—from about 5% to about 10%; lauric acid—from about 44% to about 53%; myristic acid—from about 13% to about 21%; palmitic acid—from about 5% to about 13%; stearic acid—up to about 4%; oleic acid—from about 5% to about 12%; and linoleic acid up to about 3%.

The person having ordinary skill in the art (“PHOSITA”) will understand that ingredients with the “cocoyl” name may have of variations of the above fatty acid distribution, that fatty acids in “cocoyl” moieties may be fractionated to isolate or enrich specific fractions (e.g., C₁₂ lauroyl from coconut or palm kernel fatty acids), and may be partially-or fully-hydrogenated to minimize or eliminate unsaturation (i.e. double bonds in the fatty acid chain). Accordingly, as explained by the CIR Expert Panel, sodium methyl lauroyl taurate may, and likely does contain, some sodium methyl cocoyl taurate. Similarly since myristoyl taurates have some shorter (lauroyl) and longer (palmitoyl) chain lengths, there is likely some sodium methyl lauroyl taurate in sodium methyl myristoyl taurate.

Salts of C₈-C₁₈ saturated acyl N-alkyl taurates are selected from the group consisting of sodium, potassium, magnesium, sodium taurine, sodium methyltaurine, and triethanolamine; with sodium salts being preferred.

Preferred C₈-C₁₈ saturated acyl N-alkyl taurate salts are selected from the group consisting of: magnesium methyl cocoyl taurate; potassium methyl cocoyl taurate; sodium caproyl methyltaurate; sodium methyl cocoyl taurate; sodium methyl lauroyl taurate; sodium methyl myristoyl taurate; sodium methyl palmitoyl taurate; sodium methyl stearoyl taurate; sodium methyltaurine cocoyl methyltaurate; and sodium taurine cocoyl methyltaurate.

In one particularly preferred embodiment, the C₈-C₁₈ saturated acyl N-alkyl taurate salt is a sodium salt of the coconut fatty acid amide of N-methyltaurine-namely, sodium methyl cocoyl taurate (“SMCT”), commercially available from Vantage Specialty Ingredients (Warren, NJ) as METAUPON® KMT).

SMCT is preferably present in SCs of the present invention on an active matter basis (referred to below “active basis”) at a concentration of from about 5% to about 15%, more preferably from about 6% to about 14%, and even more preferably from about 7.5% to about 12.5%.

In certain embodiments, SMCT is present in a SC of the present invention on an active basis at a concentration of at least about 5%; in other embodiments, at a concentration of at least about 6%; and in still others at a concentration of at least about 8%.

In further embodiments, SMCT is present in a SC of the present invention on an active basis at a concentration of not more than 14%; and in still further embodiments at a concentration of not more than about 12%.

An optional, but preferred, component of a SC in accordance with the first, second or third aspect of the present invention is a second acyl N-methyl taurate that is a C₁₆-C₂₂ unsaturated or branched acyl N-alkyl taurate (or salt thereof) conforming to the structure

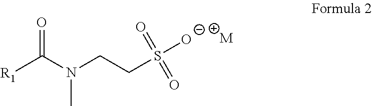

wherein R₁CO is a C₁₆-C₂₂ unsaturated or branched acyl moiety.

Preferred C₁₆-C₂₂ unsaturated or branched acyl N-alkyl taurate salts include: sodium methyl oleoyl taurate, sodium methyltaurate isopalmitamide, sodium N-isostearoyl methyltaurate.

In one particularly preferred embodiment, the salt of an unsaturated or branched acyl N-alkyl taurate is the sodium salt of the oleic acid amide of N-methyl taurine—namely, sodium methyl oleoyl taurate (“SMOT”), commercially available from Vantage Specialty Ingredients (Warren, NJ) as METAUPON® OMT.

In certain embodiments, SMOT is present in SCs of the present invention on an active basis at a concentration of from about 0.5% to about 5%; and in others at a concentration of from about 1% to about 4%.

In still other embodiments, the concentration of SMOT on an active basis in SCs of the present invention is at least about 0.5%, at least about 0.75%, at least about 1%, at least about 1.5%, at least about 2%, or at least about 3%.

A second required component of a SC in accordance with the first, second or third aspect of the present invention is a C₈-C₁₈ saturated acyl N-alkyl aminoalkanoate (or salt thereof) conforming to the structure

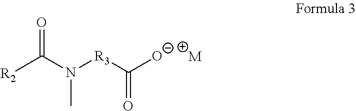

where R₂CO is a C₈-C₁₈ acyl moiety and R₃ is (CH₂)ₙ, where n is an integer from 1-5. In preferred embodiments, n is 1 or 2.

The C₈-C₁₈ saturated acyl N-alkyl aminoalkanoate is preferably a sarcosinate, a N-methyl aminopropionate or a N-methyl beta-alaninate.

Salts of C₈-C₁₈ saturated acyl N-alkyl aminoalkanoate are preferably selected from sodium, potassium, and triethanolamine; with sodium salts being preferred.

The sarcosinate may be, and preferably is, selected from the group consisting of ammonium cocoyl sarcosinate; ammonium lauroyl sarcosinate; potassium cocoyl sarcosinate; potassium lauroyl sarcosinate; sodium cocoyl sarcosinate; sodium lauroyl sarcosinate; sodium myristoyl sarcosinate; sodium oleoyl sarcosinate; and sodium palmitoyl sarcosinate.

The acyl N-alkyl aminoalkanoate may also be selected from the group consisting of sodium cocoyl methylaminopropioate; sodium lauroyl methylaminopropioate; sodium cocoyl methyl beta-alaninate; and sodium lauroyl methyl beta-alaninate.

In one particularly preferred embodiment, the salt of a C₈-C₁₈ saturated acyl N-alkyl aminoalkanoate is sodium lauroyl sarcosinate-the sodium salt of lauroyl sarcosine, which is the N-lauroyl derivative of N-methylglycine. Typically, the fatty acid profile of lauroyl sarcosine is: C₁₀—from 0 to about 2%; C₁₂—about 95%; C₁₄—about 3%; C₁₆—from 0 to about 1%; and oleic acid from 0 to about 1%.

In embodiments of the present invention in which the N-alkyl aminoalkanoate is a sarcosinate, preferably sodium lauroyl sarcosinate, the sarcosinate is present in SCs on an active basis at a concentration of from about 12% to about 30% on an active basis, preferably at a concentration of at least about 15% on an active basis, more preferably at a concentration of at least about 16% on an active basis, and most preferably at a concentration of at least about 18% on an active basis.

In especially preferred embodiments of the present invention, the SC of the present invention consists essentially of, or consists of, (a) two acyl N-methyl taurates—a first acyl N-methyl taurate that is a salt of a C₈-C₁₈ saturated acyl moiety, preferably SMCT; and a second acyl N-methyl taurate that is a salt of a C₁₆-C₂₂ unsaturated or branched acyl N-alkyl taurate, preferably SMOT; and (b) a C₈-C₁₈ saturated acyl N-alkyl aminoalkanoate, preferably a sarcosinate, more preferably sodium lauroyl sarcosinate.

The molar ratio of acyl N-alkyl aminoalkanoate to acyl N-alkyl taurate(s) in SCs of the present invention is from about 1:1 to about 4:1.

In certain embodiments, the molar ratio of acyl N-alkyl aminoalkanoate to acyl N-alkyl taurate(s) is from about 1.1:1 to about 3:1.

In other embodiments, the molar ratio of acyl N-alkyl aminoalkanoate to acyl N-alkyl taurate(s) is from about 1.5:1 to about 2.5:1 .

In preferred embodiments, the molar ratio of acyl N-alkyl aminoalkanoate to acyl N-alkyl taurate(s) is from about 1.8:1 to about 2.5:1 .

The loading (concentration) on an actives basis of surfactants in the SC-namely, the at least one, preferably, two acyl N-methyl taurates (a first acyl N-methyl taurate that is a salt of a

C₈-C₁₈ saturated acyl moiety, preferably SMCT; and a second optional but preferred acyl N-methyl taurate that is a salt of a C₁₆-C₂₂ unsaturated or branched acyl N-alkyl taurate, preferably SMOT) and the C₈-C₁₈ saturated acyl N-alkyl aminoalkanoate, preferably sodium lauroyl sarcosinate-is of from about 24% to about 40%.

In certain embodiments, the concentration of surfactants (on an active basis) in the SC is at least about 25%, at least about 26%, at least about 27%, at least about 28%, or at least about 29%.

In other embodiments, the concentration of surfactants (on an active basis) in the SC is at least about 30%.

In still other embodiments, the concentration of surfactants (on an active basis) in the SC is at least about 32%

In yet other embodiments, the concentration of surfactants (on an active basis) in the SC is at least about 40%.

SCs of the present invention are substantially free of (i) surfactants comprising a sulfate moiety and (ii) surfactants comprising a hydrogen bond donor that is an amide (also referred to below hydrogen bond donating amide moiety). The substantial absence of a surfactant having either a sulfate moiety or a hydrogen bond donating amide moiety is a novel and basic property of the SC of the present invention.

Anionic, cationic, or zwitterionic (amphoteric/betaine) surfactants having at least one hydrogen bond donating amide moiety (e.g., a secondary amide) are not included in the SC of the present invention.

Anionic surfactants having least one hydrogen bond donating amide moiety conform the following structure

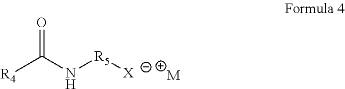

Non-limiting examples of anionic surfactants according to Formula 4 include: N-acyl taurates (e.g., sodium cocoyl taurate, sodium lauroyl taurate, potassium cocoyl taurate); N-acyl glycinates (e.g., sodium cocoyl glycinate, sodium lauroyl glycinate); N-acyl beta-alaninates (e.g., sodium lauroyl beta-alaninate), N-acyl alaninates (e.g. sodium cocoyl alaninate), and N-acyl glutamates (e.g., sodium cocoyl glutamate).

Zwitterionic surfactants having least one hydrogen bond donating amide moiety conform the following structure

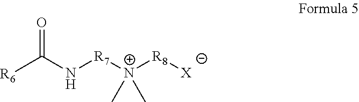

Non-limiting examples of zwitterionic according to Formula 5 include: N-acyl amidopropyl betaines (e.g., cocamidopropyl betaine; lauramidopropyl betaine); and N-acyl amidopropyl hydroxysultaines (cocamidopropyl hydroxysultaine, lauramidopropyl hydroxysultaine).

SCs of the present invention are “freely-flowable” by which is meant having a viscosity of less than about 1,000 cps, preferably less than about 500 cps, more preferably less than about 300 cps, and even more preferably less than about 100 cps, as measured with a viscometer. Spindle and rotation speed settings on a viscometer are selected to ensure viscosity readings are taken within a torque window of 10-80% of the maximum torque range. In the examples below, the viscosities of SCs are recorded with a Brookfield™ viscometer (Ametek, Inc., Berwyn, PA) using LV-2 spindle at a rotation speed of 60 rpm at room temperature (from about 20° C. to about 25° C.) and ambient conditions (about 1 atmosphere of pressure and at about 50% relative humidity).

SCs of the present invention preferably have a pH of greater than about 6.5. Decreasing pH of a SC below 6.5 can result in an increased viscosity which is undesirable (e.g, rendering the SC not “freely flowable”). Without wishing to be bound by theory, Applicant believes such an increase in viscosity may be attributed to protonation of the acyl N-alkyl aminoalkanoate carboxylate group, which induces micellar thickening by formation of entangled worm-like micelle structures in the surfactant concentrate.

SCs of the present invention exhibit substantial transparency or translucency, which can be measured by visual observation or by instrumentation known to the PHOSITA, including by using a clarity meter, a spectrophotometer, or a turbidimeter. When measured with a turbidimeter in accordance with ISO Test Method 7027-1:2016, SCs of the present invention have a measured turbidity expressed in Nephelometric Turbidity Units (NTUs) of less than about 10 NTU, preferably less than about 8 NTU, more preferably less than about 6 NTU, still more preferably less than about 5 NTU, even more preferably less than about 3 NTU, and most preferably less than about 2 NTUs, indicating clarity and absence of haze. This property allows formulators to create substantially transparent or translucent finished goods-namely, cleansing and cleansing-conditioning products for hair, skin, and scalp.

In preferred embodiments, being freely flowable (viscosity of less than 1,000 cps) and having substantial transparency/translucency (measured turbidity of less than about 10 NTU) are required elements of the SCs of the present invention; they are novel and basic properties of the inventive SCs. Additional novel and basic properties of SCs of the present invention are described below.

Another basic and novel property of the SC of the present invention is self-preservation—no additional preservative ingredients are required to be added to the inventive concentrate to pass the Personal Care Products Council (PCPC) microbial testing guidelines, for example PCPC M-2 (testing for Staphylococcus aureus, Escherichia coli and Pseudomonas aeruginosa).

Yet another basic and novel property of concentrates according to the present invention is the ability to be used in forming stable, cold process, stable emulsions. By “cold process” is meant that in the presence of SCs of the present invention aqueous and non-aqueous components can be combined (mixed) to form a stable emulsion without the need to heat the mixture. By “stable” is meant an emulsion that does not separate into aqueous and non-aqueous phases after three freeze/thaw cycles and/or after accelerated storage testing at 45° C. for three months.

A fourth aspect of the present invention is directed to a method for making a finished product (cleanser or combination 2-in-1 cleanser-conditioner) for hair, scalp, or skin (body or face) comprising the step of adding a SC of the first, second or third aspects of the invention.

The combined surfactant contribution (load) in a finished formulation on an active basis from a SC (alone) in accordance with any of the first, second or third aspects of the invention is preferably from about 3% to about 20%, more preferably from about 6% to about 18%, and even more preferably from about 9% to about 15%. In certain embodiments, the surfactant load from a SC (alone) on an active basis may be from about 3% to about 9%. In other embodiments, the surfactant load from a SC (alone) on an active basis may be from about 6% to about 15%.

While a SC of present invention is a complete surfactant system (i.e., no additional surfactant(s) is/are required to achieve desired performance), one (or more) additional surfactant(s) may be added to a finished product (e.g., to enhance thickening or to solubilize fragrances or oils).

In some of embodiments where a finished product contains one (or more) additional surfactant(s) (beyond a SC of the present invention), the additional surfactant(s) is/are preferably substantially free of a sulfate moiety.

In certain embodiments, a finished product is made by the step of adding one or more additional surfactant(s) (beyond a SC) selected from the group consisting of non-ionic, anionic, cationic, and zwitterionic surfactants, including an anionic or zwitterionic surfactant having least one hydrogen bond donating amide moiety as described in Formulas 4 and 5.

Preferably, the one or more additional surfactant(s) is/are zwitterionic and/or non-ionic.

In embodiments containing one or more additional surfactants, the total concentration of surfactants (on an active basis) is preferably less than 25% by weight of the finished product.

A cleanser or cleanser-conditioner made in accordance with the fourth aspect of the present invention may and preferably does contain one or more ingredients known to a PHOSITA, including pH adjusters, chelating agents, skin conditioning agents, hair conditioning agents, humectants, emollients, rheology modifiers, abrasive particles (e.g., as exfoliants), opacifiers, colorants, preservatives, fragrances, and sensates, non-limiting examples of which are disclosed in U.S. Pat. No. 9,993,408, and incorporated by reference herein in its entirety.

Cleansers or cleanser-conditioners made in accordance with the fourth aspect of the present invention may, for example, contain a multifunctional, jojoba-derived sensorial enhancer that provides emolliency, moisturizing, softening and conditioning benefits (among others) and consists essentially of or consists of (i) from about 75 to about 85 parts hydrolyzed jojoba esters (formed by reaction of jojoba oil with an alkali metal hydroxide in water), (ii) from about 8 to about 15 parts jojoba esters, and (iii) from about 7 to about 10 parts water, which is commercially available under the tradename LIPONATE® NATFILM® from Vantage Specialty Ingredients, Inc. (Warren, NJ), referred to hereinbelow as “NF”.

NF is preferably present in cleansers or cleanser-conditioners at a concentration of from about 0.1 wt-% to about 5 wt-%, preferably at a concentration from about 0.5 wt-% to about 3 wt-%, based on the total weight of the finished product. In such finished products, NF may be, and preferably is, combined with glycerin, in a ratio of glycerin to NF of from about 5:1 to about 2:1. In these embodiments, NF is present in the composition at a concentration of at least about 0.1%, preferably from about 0.2% to about 3.0% by weight of the finished formulation; and glycerin is present in the composition at a concentration of at least about 0.1%, preferably from about 0.4% to about 6% by weight of the finished formulation.

Cleansers or cleanser-conditioners made in accordance with the fourth aspect of the present invention provide one or more of the following benefits: easier combability (dry and wet); reduced frizz/volume control; reduced hair breakage; reduced split ends; increased hair fiber strength; increased luster/shine; reduced hair cuticle damage; improved hair surface quality (shine and smoothness); improved skin hydration; improved skin barrier function/reduced trans-epidermal water loss.

A fifth aspect of the present invention is a method of imparting one or more benefits to hair or skin comprising the step of applying to the skin, hair or scalp a cleanser or cleanser-conditioner made in accordance with the fourth aspect of the present invention. The one or more benefits is selected from the group consisting of easier combability (dry and wet); reduced frizz/volume control; reduced split ends; increased hair fiber strength; increased luster/shine; reduced hair cuticle damage; improved hair surface quality (shine and/or smoothness).

The following test methods are known to PHOSITA and may be used to demonstrate the benefits provided by a cleanser or cleanser-conditioner made in accordance with the fourth or fifth aspects of the present invention.

Foamability including quality of lather (foam stability, foam height, bubble size and bubble distribution) can be measured instrumentally, for example using Dynamic Foam Analyzer from Krüss Scientific (Hamburg, Germany).

Additional methods assessing quality of foam known and used by persons having ordinary skill in the art include the “cylinder shake” method. In accordance with this method, a fixed amount of cleaning product (shampoo or body wash) is poured into a graduated cylinder; a stopper is placed onto the cylinder, which is inverted a fixed number of times; foam volume is then measured.

The cylinder shake foam test can be modified and “standardized” to reduce operator dependence and thus more reliably emulate consumer experience. In one variation, 300 ml of dilute surfactant solution are added to a 1,000 ml graduated cylinder. The cylinder is rotated on a vertical plane perpendicular to the axis of a motor attached to the cylinder (e.g., for 2 minutes at 36 rpm). Foam height reading is taken 30 seconds after rotation has finished.

In another variation, a 10% solution of dilute surfactant solution is prepared. Four grams of the solution are added to 146 grams of water (50 ppm hardness) at 29° C. The solution is agitated for 10 seconds at a medium speed in a blender. The foam is poured into a 100 ml graduated cylinder until overflowing. A rubber stopper-slightly smaller in diameter than the inside diameter of the graduated cylinder-is gently dropped into the foam. The time for the rubber stopper to pass between two points (80 ml-40 ml) is measured. A longer time indicates a denser and more stable foam and closely approximates consumer perception of foam “quality.”

In place of a blender, a stir bar can be used to generate foam; after which foam height is measured over a set time period. This method was used in Applications Example 3 below.

Improved hair surface quality and reduced cuticle damage from use of the complexes of the present invention disclosure can be assessed using scanning electron microscopy and/or on a five-point scale from 1 (most damage; lowest score) to 5 (highest score; least damage): (1)—missing sections of cuticle; large “blocks” of scales dislodged or uplifted; (2)—significant uplift, cracking, chipping and other signs of wear of individual scales; fragmented and dislodged cuticle material visible on surface; (3)—some uplift, cracking and fragmentation of individual scales; (4)—small amount uplift and minor wear and tear at cuticle edges; (5)—minimal wear and tear at cuticle edges; de minimus to no uplift. See, Cosm & Toil 132(4)38-48 (April 2017).

Cuticle damage can be caused by expansion and contraction each time the hair is washed and dried. This damage can be exacerbated by friction between adjacent fibers during brushing/combining. Without being bound by a theory, applicant believes that the complexes of the present invention disclosure prevent weakening or degradation of the inter-cuticular “cement” between the cuticle scales.

Improved hair surface quality from use of the complexes of the present invention disclosure is expressed in terms of shine/luster and can be quantified as follows: Light reflectance is described as either “specular” (at a ninety-degree angle to incident light) or “diffuse” (at an angle not ninety degrees). Specular reflectance is greater from smooth versus rough surfaces. The greater the amount of specular reflectance (or reflectance close to ninety degrees) the higher degree of what is perceived as shine or luster. Hair shine can be determined by self-reporting or trained observation, but also by quantification. Improvement in hair shine can be measured with polarizing filters in a two-step procedure. First, two polarized filters are mounted in parallel configuration, capturing total reflected light (specular reflection and diffuse reflection). Second, perpendicular polarized filters are used, eliminating specular reflectance, and capturing only diffuse reflectance. Specular reflectance is calculated as the difference between the first and second measurements. See, Cosm & Toil 131(1)28-34 (January/February 2016).

Shine can be measured with a gloss meter, for example YG60S 60° Economic Gloss Meter (Shenzhen 3nh Technology Co., Ltd.). Light is projected onto a standardized smooth surface and reflectance quantified at 85° which serves as a non-dimensional reference value (“zero”) of relative gloss (shine). Additional measurements are taken at angles of 20° and 60°.

Another methodology for measuring improved hair shine from use of the complexes of the present invention disclosure employs a goniophotometer (“GP”). Samples of hair strands of different ethnicities and shades with no known history of chemical treatments (e.g., blond Piedmont hair, light and dark brown European hair, black Indian hair, black Japanese hair, black African-American hair, black Chinese hair). For each hair type, measurements were made on 25 randomly chosen fibers. Samples are mounted horizontally and illuminated with standardized light sources (e.g., He-Ne light source (632-nm) and quartz tungsten halogen lamp emitting white light). Measurements are performed in the root-to-tip direction at an angle of incidence of 45°. Reflected light is detected as a function of angle. Using peak-fit software, the GP curve is separated into specular and diffuse components. Luster (shine) is calculated by the equation: L=S/((S+D) where Sis defined as the specular peak area obtained from the scattering curve and (S+D) is the total area under the curve. See, J. Soc. Cosmet. Chem., 44, 221-234 (July/August 1993).

Reduced hair breakage from use of the complexes of the present invention disclosure can be measured as follows: Tensile properties of hair (e.g., strength, stiffness) relate to the structure of the inner cortex of the follicle. Reduced damage to the inner cortex of hair fibers is demonstrated indirectly as a function of the hair's tensile properties. Other factors being equal, thick hair fiber is stronger than thin fiber. Increased fiber diameter is measured using image analysis techniques (e.g., photomicroscopy) known to the person having ordinary skill in the art.

Hair fatigue can result from repeated grooming (combining/brushing). Hair strength is a measurement of resistance to stress (i.e., applied force divided by cross-sectional area of hair fiber) and is expressed as “force-to-break,” represented graphically in a stress-stain plot, with percentage extension on the x axis, and force on the y axis. See Cosm. & Toil. 13 (6)36-45 (June 2017). The ability of complexes of the present invention disclosure to reduce hair fatigue (damage to the inner cortex of hair fibers) and thus increase hair strength is expressed in an S-N Plot in which the magnitude of the repeating applied stress (S) is plotted against the number of cycles (N) required to induce breakage. Reduced breakage is associated with increased hair strength. See J Cosmet Sci. 2009 November-December;60(6):599-616; and J Cosmet Sci. 2010 November-December;61(6):439-55. See also, Cosm & Toil 128(8)590-594 (August 2013) and Cosm & Toil 128(12) 854-859 (December 2013).

The ability of compositions of the present invention to reduce skin (and scalp) dryness can be assessed in terms of moisture content at several time points: baseline; immediately after application; post application (e.g., at 4-, 6- or 8-hours post-application); and, optionally, 2 hours post-application. Skin moisture content can be measured in terms of capacitance, conductance, or impedance using instruments known to the person having ordinary skill in the art including but not limited to a Corneometer. Maintenance of increased moisture for longer durations is indicative of superior efficacy in terms of reducing skin dryness.

Increased (e.g., improved) skin barrier function can be assessed in terms of trans-epidermal water loss (TEWL) using a Tewameter, which measures the rate of evaporation from the skin surface. In accordance with the present invention disclosure, TEWL is measured on a test site (e.g., 2 cm2) marked on the lower lateral leg as follows. At least three Tewameter measurements are taken: (i) prior to stripping (removal of the stratum corneum, resulting in increased TEWL); (ii) post-stripping; (iii) post application of the test product. Return of TEWL to a level approximately the same as baseline is indicative of improved skin barrier function. Skin dryness can also be graded on a visual analog scale from 0 to 10, where “0” represents no or very little evidence of dryness, and “10” which represents severe flaking, peeling and/or fissures.

Irritation can be observed or measured with clinical instrumentation as redness (erythema) or measured in vitro, including by testing the solubility of zein, a corn protein similar to keratin in human hair and skin in the presence of surfactants. Zein is insoluble in water unless it is denatured. In the zein test, protein denaturation is used as a predictor of skin irritation; increasing solubility of zein correlates with increased skin irritancy. Götte. Skin compatibility of tensides measured by their capacity for dissolving zein protein. In: Proc IV International Congress on Surface Active Substances, Brussels, Belgium, pp. 83-90 (1964). See also, Morrison Jr, B. M. and M. Paye, A comparison of three in vitro screening tests with an in vivo clinical test to evaluate the irritation potential of. J Soc Cosmet Chem, (1995) 46:291-299.

On an active basis, concentrates of the present invention have a lower zein score than sulfate surfactant systems having the same active basis.

### EXAMPLES

The following examples are illustrative. Modifications will be apparent to, and can be readily made by, those skilled in the art without departing from the spirit and scope of the invention. The scope of the appended claims is not to be limited to the examples.

### Sulfate-Free Surfactant Concentrate of the Present Invention

| Surfactant | Activity % | SC-1 | SC-2 | SC-3 | SC-4 |
|---|---|---|---|---|---|
| SMCT | 30 | 25.00 | 30.33 | 32.53 | 34.11 |
| SMOT | 75 | 3.00 | 3.65 | 4.30 | 0.00 |
| Sodium lauroyl | 30 | 48.40 | 58.73 | 63.17 | 65.89 |
| sarcosinate | | | | | |
| Water | 100 | 23.60 | 7.29 | — | 0.00 |
| pH | N/A | 6.88 | 6.91 | 7 | 6.95 |
| Viscosity (cps) | N/A | 23 | 23 | 102.5 | 23.5 |
| Turbidity (NTU) | N/A | 0.85 | 1.41 | 1.38 | 1.56 |

| | Active | [Sarcosinate]:[N-methyl |
|---|---|---|
| Example | surfactant (wt %) | taurate] mol ratio |
| SC-1 | 24 | 1.9 |
| SC-2 | 29 | 1.9 |
| SC-3 | 32 | 1.9 |
| SC-4 | 30 | 2.4 |

### Comparative Sulfate-Free Surfactant Concentrates

| Surfactant | Activity % | Comp-1 | Comp-2 | Comp-3 | Comp-4 | Comp-5 |
|---|---|---|---|---|---|---|
| SMCT | 30 | 32.53 | 32.53 | 34.11 | 34.11 | 32.53 |
| SMOT | 75 | 4.30 | 4.30 | — | — | 4.30 |
| Sodium lauroyl | 30 | — | — | — | — | — |
| sarcosinate | | | | | | |
| Sodium lauroyl | 94 | — | — | — | — | — |
| sarcosinate | | | | | | |
| Sodium Cocoyl | 31 | 63.17 | — | 65.89 | — | — |
| Glycinate | | | | | | |
| Cocamidopropyl | 49 | — | 40.00 | — | 40.35 | — |
| Hydroxysultaine | | | | | | |
| Sodium Cocoyl | 30 | — | — | — | — | 63.17 |
| Alaninate | | | | | | |
| Water | 100 | — | 23.17 | — | 25.54 | — |
| pH | N/A | 9.3 | 6.7 | 9.34 | 6.66 | 7.7 |
| Viscosity (cps) | N/A | 92,500 | 223,750 | 3,800 | 62,500 | 3,900 |
| Turbidity (NTU) | N/A | 1,283 | 17.7 | 648 | 12.9 | 510 |

The comparative examples, which replace a C₈-C₁₈ saturated acyl N-alkyl aminoalkanoate with a surfactant bearing a hydrogen bond donating amide moiety, are neither free-flowing nor substantially transparent or translucent.

### Formulation Examples

### Cleansing Compositions with Sulfate and Sulfate-Free Surfactant Systems

| Phase | INCI Name | Example 1 | Example 2 |
|---|---|---|---|
| 1 | Water | 72.45 | 59.95 |
| 1 | Disodium EDTA | 0.10 | 0.10 |
| 2 | Guar Hydroxypropyltrimonium | 0.60 | 0.60 |
| | Chloride | | |
| 3 | Water (and) Citric Acid | 1.60 | 1.60 |
| 4 | Hydroxypropyl Methylcellulose | 1.50 | 1.50 |
| 4 | Glycerin | 5.00 | 5.00 |
| 5 | Sodium Laureth Sulfate (60% active) | 12.50 | 0.0 |
| 5 | Cocamidopropyl Betaine | 5.00 | 0.0 |
| | (30% active basis) | | |
| | SC of the Present Invention: | 0.0 | 30.00 |
| | Sodium Methyl Cocoyl Taurate | | |
| | (and) Sodium Oleoyl Cocoyl | | |
| | Taurate (and) Sodium | | |
| | Lauroyl Sarcosinate | | |
| 6 | Phenoxyethanol (and) Caprylyl | 1.00 | 1.00 |
| | Glycol (and) Hexylene Glycol | | |
| 6 | Fragrance | 0.25 | 0.25 |

### Applications Example 3

### Foamability

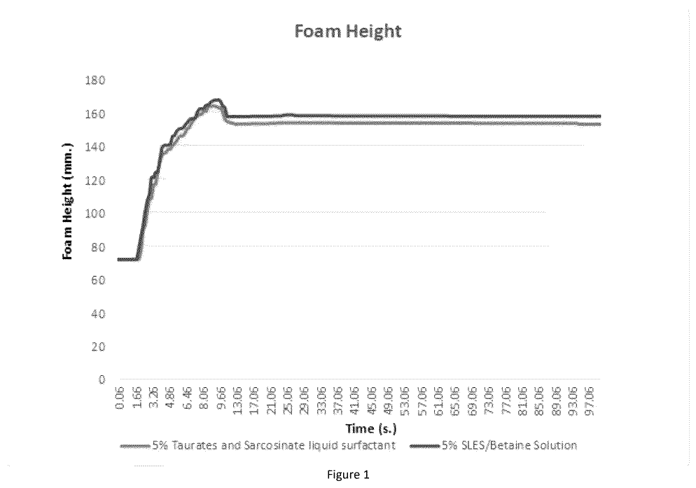

Two test products, each with a 5% surfactant system on an active basis, were prepared. The first product contained SLES and CAPB in a 1:2 ratio. The second product contained (a) sodium methyl cocoyl taurate, (b) sodium methyl oleoyl taurate and (c) sodium lauroyl sarcosinate, in a ratio a:b:c of about 3:1:6. 80 ml of the test products were mixed with a stir bar at 5,000 rpm for ten seconds to generate foam. FIGS. 1 and 2 show comparative foam height and bubble count of the two products as measured with a Krüss Dynamic Foam Analyzer.

### Applications Example 4

### Combability

Five test solutions, each with 9% surfactant(s) on active basis were tested for combability, both wet and dry: (i) SLES; (ii) SLES and CAPB in a 2:1 ratio; (iii) Sodium Cocoyl Isethionate; (iv) Decyl Glucoside; and (v) concentrate according to the present invention—namely, (a) sodium methyl cocoyl taurate, (b) sodium methyl oleoyl taurate and (c) sodium lauroyl sarcosinate, in a ratio a:b:c of about 3:1:6.

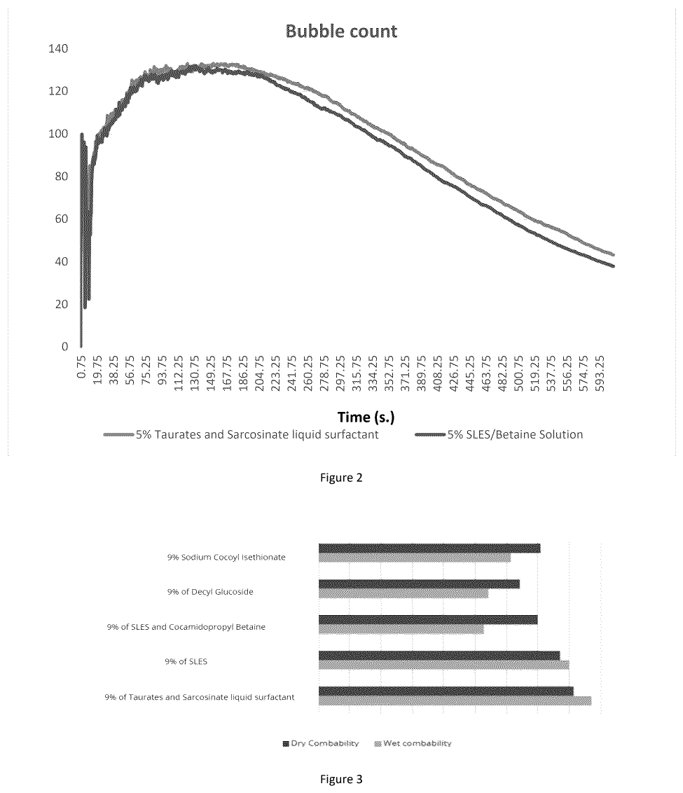

Tresses of natural Caucasian hair were massaged with 1.5 ml of a 10% SLES solution for three minutes and rinsed under water at about 36° C. for one minute; and then allowed to dry for 24 hours. Baseline glossmeter measurement were taken. The tresses were then wet for 20 seconds; excess water was removed; and the test solutions were applied (rubbed onto the tresses) for 15 seconds. After 15 seconds, the tresses were rinsed with water; and air dried for 24 hours. Combability was then evaluated by trained panelists. Results are presented in FIG. 3.

### Applications Example 5

### Frizz

A 9% test solution was prepared by adding DI water to a concentrate of the present invention-(a) sodium methyl cocoyl taurate, (b) sodium methyl oleoyl taurate and (c) sodium lauroyl sarcosinate, in a ratio a:b:c of about 3:1:6. Two tresses of hair were prepared (washed and dried) in the manner described in Example 4. A first tress was wet for 20 seconds; excess water was removed; and the test solutions was applied (rubbed onto the tress) for 15 seconds. After 15 seconds, the first tress was rinsed with water; and air dried for 24 hours. The second tress was not treated. After the first tress air dried, both tresses were placed in a 75-80% humidity-controlled room. The width of each tress was then measured at five time points: baseline (initial); 1 hour; 2 hours; 6 hours; and 24 hours.

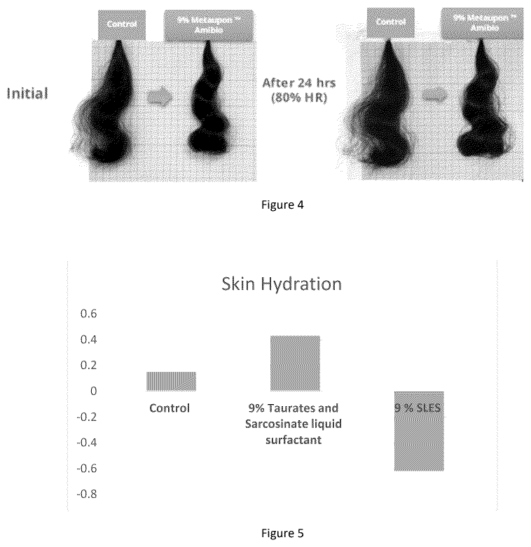

Compared to the control, the 9% solution containing concentrate of the present invention reduced frizz by 35% at the initial measurement and by 25% after 24 hours. Results are presented in FIG. 4.

### Applications Example 6

### Skin Hydration

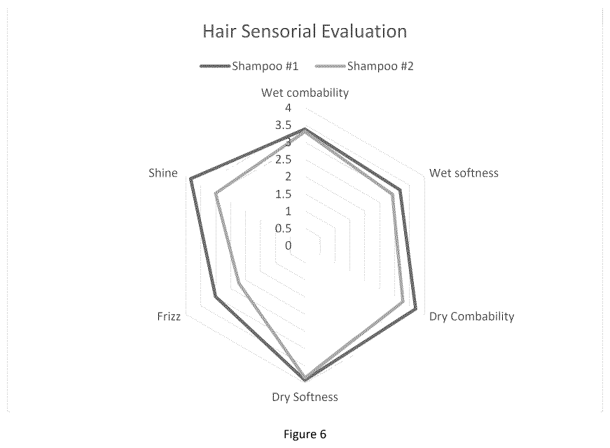

A test area—3cm² on the forearm of a volunteer—was washed a 10% SLES solution (active basis) and dried. Two test solutions were prepared; the first solution contained a 9% of the concentrate of the present invention (active basis)—(a) sodium methyl cocoyl taurate, (b) sodium methyl oleoyl taurate and (c) sodium lauroyl sarcosinate, in a ratio a:b:c of about 3:1:6; the second solution contained 9% SLES (active basis). After a baseline hydration measurement was taken with a Corneometer® CM 825 (Courage+Khazaka electronic GmbH, Köln, Germany), 0.2 ml of each test solution was applied and rubbed on the test area 30 seconds and then rinsed off. This procedure was repeated 3 times. After the third cycle, measurements Corneometer measurements were repeated. Results are presented in FIG. 5.

### Applications Example 7

### Hair Sensorial Evaluation

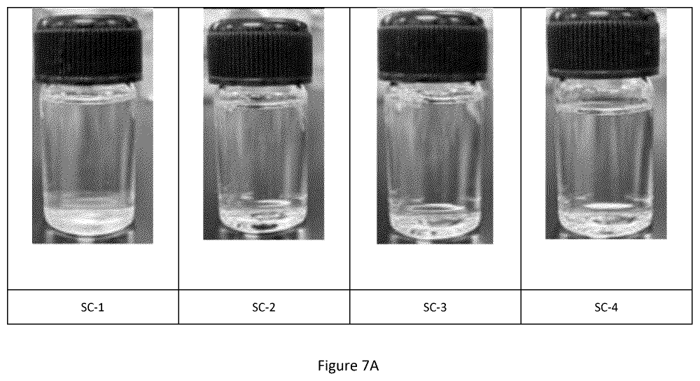

Tresses of curly hair were massaged with 1.5 ml of a 10% SLES solution for three minutes and rinsed under water at about 36° C. for one minute; and then allowed to dry for 24 hours. The tresses were wet for 20 second. Two test formulations-corresponding to Examples 1 and 2 (respectively Shampoos 1 and 2)-were applied and massed on the tresses for 30 seconds; and then rinsed. 10 trained panelists evaluated the tresses before and after application of the test formulations for combability (wet and dry), softness (wet and dry), shine and frizz. Results are presented in FIG. 6.

The articles “a”, “an”, and “the” are to be understood to mean “one or more.” All numeric ranges are inclusive of narrower ranges and combinable; delineated upper and lower range limits are interchangeable to create further ranges not explicitly delineated. The number of significant digits conveys neither a limitation on the indicated amounts nor on the accuracy of the measurements. All numerical amounts are to be understood as modified by the word “about”.

1. A surfactant concentrate comprising
a. at least one, preferably two, acyl N-alkyl taurate(s), or salt(s) thereof;
b. an acyl N-alkyl aminoalkanoate, or salt thereof; and
c. water
wherein the molar ratio of the acyl N-alkyl aminoalkanoate to the acyl N-alkyl taurate(s) is from about 1:1 to about 3:1; and
wherein the surfactant concentrate is substantially free of any surfactant that is comprised of (i) a sulfate moiety and/or (ii) a hydrogen bond donating amide moiety.

2. A surfactant concentrate consisting essentially of
a. at least one, preferably two, acyl N-alkyl taurate(s), or salt(s) thereof;
b. an acyl N-alkyl aminoalkanoate, or salt thereof; and
c. water
wherein the molar ratio of the acyl N-alkyl aminoalkanoate to the acyl N-alkyl taurate(s) is from about 1:1 to about 3:1; and
wherein the surfactant concentrate is substantially free of any surfactant that is comprised of (i) a sulfate moiety and/or (ii) a hydrogen bond donating amide moiety.

3. The surfactant concentrate of claim 1 or 2 wherein the at least one acyl N-alkyl taurate (or salt thereof) is a saturated acyl N-alkyl taurate conforming to the structure 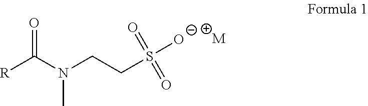
where RCO is a C₈-C₁₈ acyl moiety.

4. The surfactant concentrate of claim 3 wherein the at least one acyl N-alkyl taurate (or salt thereof) is sodium methyl cocoyl taurate.

5. The surfactant concentrate of any of claims 1-4 containing a second acyl N-alkyl taurate (or salt thereof) that is an unsaturated or branched acyl N-alkyl taurate (or salt thereof) conforming to the structure 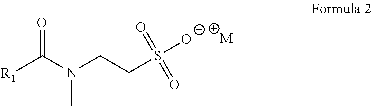
wherein R₁CO is a C₁₆-C₂₂ acyl moiety.

6. The surfactant concentrate of claim 5 wherein the second acyl N-methyl taurate is sodium methyl oleoyl taurate.

7. The surfactant concentrate of any of claims 1-6 wherein the acyl N-alkyl aminoalkanoate (or salt thereof) is a saturated acyl N-alkyl aminoalkanoate (or salt thereof) conforming to the structure 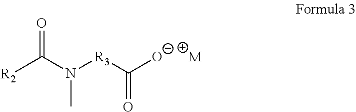
where R₂CO is a C₈-C₁₈ acyl moiety and R₃ is (CH₂)ₙ, where n is an integer from 1-5.

8. The surfactant concentrate of claim 7 wherein n is 1 or 2.

9. The surfactant concentrate of any of claims 1-8 wherein the acyl N-alkyl aminoalkanoate (or salt salt thereof) is sodium lauroyl sarcosinate.

10. The surfactant concentrate of claim 9 wherein the molar ratio of acyl N-alkyl aminoalkanoate to acyl N-alkyl taurate(s) is from about 1.5:1 to about 2.5:1

11. The surfactant concentrate of claim 10 wherein the molar ratio of acyl N-alkyl aminoalkanoate to acyl N-alkyl taurate(s) is from about 1.8:1 to about 2.5:1

12. The surfactant concentrate of any of claims 1-11 having a pH of greater than about 6.5.

13. The surfactant concentrate of claim 12 having a viscosity of less than about 1,000 cps as measured with a Brookfield viscometer (LV-2 spindle at 60 rpm) at room temperature and ambient conditions.

14. The surfactant concentrate of any of claims 13-15 wherein the total concentration of surfactants on an active basis is at least about 24%.

15. The surfactant concentrate of claim 16 wherein the total concentration of surfactants on an active basis is at least about 30%.

16. The surfactant concentrate of claim 17 wherein the total concentration of surfactants on active basis is at least about 32%.

17. The surfactant concentrate of any of claims 16-18 that is substantially transparent or translucent based on a turbidity measurement of less than about 10 NTU using ISO Test Method 7027-1:2016.

18. The surfactant concentrate of claim 19 having a turbidity measurement of less than about 2 NTU using ISO Test Method 7027-1:2016.

19. A method of making a surfactant concentrate comprising the step of combining (a) at least one, preferably two, acyl N-alkyl taurate(s), or salt(s) thereof and (b) an acyl N-alkyl aminoalkanoate, or salt thereof in a molar ratio a:b of from about 1:1 to about 3:1.

20. A method of making a cleanser or cleanser-conditioner comprising the step of combining a surfactant concentrate according to any of claims 1-20 with one or more additional surfactant(s), with the proviso that the additional surfactant(s) are substantially devoid of a sulfate moiety.

21. The method of claim 22 wherein the one or more additional surfactant(s) is/are non-ionic or zwitterionic.

22. The method of any of claim 22 or 23 wherein total surfactants (on an active basis)—in the surfactant concentrate according to any of claims 1-20 and in the one or more additional surfactant(s)—are present in the cleanser or cleanser-conditioner at a concentration of less than about 25 wt %.

23. A cleanser or cleanser-conditioner for application to the hair, scalp or skin of a mammal comprising a surfactant concentrate of any of claims 1-20 and one or more ingredients from the group consisting of pH adjusters, chelating agents, skin conditioning agents, hair conditioning agents, humectants, emollients, rheology modifiers, abrasive particles, opacifiers, colorants, preservatives, fragrances, and sensates wherein the cleanser or cleanser-conditioner is comprised of less than about 25 wt % surfactants on an active basis.

24. A method of imparting one or more benefits to hair or skin comprising the step of applying to the skin, hair or scalp a cleanser or cleanser-conditioner according to claim 25 wherein the one or more benefits is selected from the group consisting of easier combability (dry and wet);
reduced frizz/volume control; reduced split ends; increased hair fiber strength; increased luster/shine; reduced hair cuticle damage; improved hair surface quality (shine and/or smoothness).
